(19) United States
(12) Patent Application Publication
(10) Pub. No.: US 2008/0183237 A1
(43) Pub. Date: Jul. 31, 2008

(54) Methods And Apparatus For Treating Ileus Condition Using Electrical Signals

(75) Inventors: Joseph P. ERRICO, Green Brook, NJ (US); Steven MENDEZ, Chester, NJ (US); Peter S. STAATS, Colts Neck, NJ (US)

Correspondence Address: KAPLAN GILMAN GIBSON & DERNIER L.L.P., 900 ROUTE 9 NORTH, WOODBRIDGE, NJ, 07095, US

(73) Assignee: ELECTROCORE, INC., Summit, NJ (US)

(21) Appl. No.: 11/735,709

(22) Filed: Apr. 16, 2007

Related U.S. Application Data
(60) Provisional application No. 60/792,823, filed on Apr. 18, 2006.

(51) Int. Cl. A61N 1/04

(52) U.S. Cl. 607/40

(57) ABSTRACT

A method of treating a temporary arrest of intestinal peristalsis includes inducing at least one of an electric current, an electric field and an electromagnetic field in a sympathetic nerve chain of a mammal to block and/or modulate inhibitory nerve signals thereof such that intestinal peristalsis function is at least partially improved.

### CROSS REFERENCE TO RELATED APPLICATIONS

This application claims the benefit of U.S. Provisional Patent Application No: 60/792,823, filed Apr. 18, 2006, the entire disclosure of which is hereby incorporated by reference.

### BACKGROUND OF THE INVENTION

The present invention relates to the field of delivery of electrical impulses to bodily tissues for therapeutic purposes, and more specifically to devices and methods for treating conditions associated with a temporary arrest of intestinal peristalsis, such as paralytic Ileus, adynamic Ileus, and/or paresis.

The use of electrical stimulation for treatment of medical conditions has been well known in the art for nearly two thousand years. One of the most successful modern applications of this basic understanding of the relationship between muscle and nerves is the cardiac pacemaker. Although its roots extend back into the 1800's, it wasn't until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed. Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

There are two types of intestinal obstructions, mechanical and non-mechanical. Mechanical obstructions occur because the bowel is physically blocked and its contents can not pass the point of the obstruction. This happens when the bowel twists on itself (volvulus) or as the result of hernias, impacted feces, abnormal tissue growth, or the presence of foreign bodies in the intestines. Ileus is a partial or complete non-mechanical blockage of the small and/or large intestine. Unlike mechanical obstruction, non-mechanical obstruction, Ileus or paralytic Ileus, occurs because peristalsis stops. Peristalsis is the rhythmic contraction that moves material through the bowel.

Ileus may be associated with an infection of the membrane lining the abdomen, such as intraperitoneal or retroperitoneal infection, which is one of the major causes of bowel obstruction in infants and children. Ileus may be produced by mesenteric ischemia, by arterial or venous injury, by retroperitoneal or intra-abdominal hematomas, after intra-abdominal surgery, in association with renal or thoracic disease, or by metabolic disturbances (e.g., hypokalemia).

Gastric and colonic motility disturbances after abdominal surgery are largely a result of abdominal manipulation. The small bowel is largely unaffected, and motility and absorption are normal within a few hours after operation. Stomach emptying is usually impaired for about twenty four hours, but the colon may remain inert for about forty-eight to seventy-two hours (and in some cases 4-7 days). These findings may be confirmed by daily plain x-rays of the abdomen taken postoperatively; they show gas accumulating in the colon but not in the small bowel. Activity tends to return to the cecum before it returns to the sigmoid. Accumulation of gas in the small bowel implies that a complication (e.g., obstruction, peritonitis) has developed.

Symptoms and signs of Ileus include abdominal distention, vomiting, obstipation, and cramps. Auscultation usually reveals a silent abdomen or minimal peristalsis. X-rays may show gaseous distention of isolated segments of both small and large bowel. At times, the major distention may be in the colon. When a doctor listens with a stethoscope to the abdomen there will be few or no bowel sounds, indicating that the intestine has stopped functioning. Ileus can be confirmed by x rays of the abdomen, computed tomography scans (CT scans), or ultrasound. It may be necessary to do more invasive tests, such as a barium enema or upper GI series, if the obstruction is mechanical. Blood tests also are useful in diagnosing paralytic Ileus.

Conventionally, patients may be treated with supervised bed rest in a hospital, and bowel rest—where nothing is taken by mouth and patients are fed intravenously or through the use of a nasogastric tube. In some cases, continuous nasogastric suction may be employed, in which a tube inserted through the nose, down the throat, and into the stomach. A similar tube can be inserted in the intestine. The contents are then suctioned out. In some cases, especially where there is a mechanical obstruction, surgery may be necessary. Intravenous fluids and electrolytes may be administered, and a minimal amount of sedatives. An adequate serum K level (>4 mEq/L [>4 mmol/L]) is usually important. Sometimes colonic Ileus can be relieved by colonoscopic decompression. Cecostomy is rarely required.

Drug therapies that promote intestinal motility (ability of the intestine to move spontaneously), such as cisapride and vasopressin (Pitressin), are sometimes prescribed. Some reported opiate therapies (such as alvimopan) are directed to inhibiting sympathetic nerve transmission to improve intestinal peristalsis.

Alternative practitioners offer few treatment suggestions, but focus on prevention by keeping the bowels healthy through eating a good diet, high in fiber and low in fat. If the case is not a medical emergency, homeopathic treatment and traditional Chinese medicine can recommend therapies that may help to reinstate peristalsis.

Ileus persisting for more than about one week usually involves a mechanical obstructive cause, and laparotomy is usually considered. Colonoscopic decompression may be helpful in cases of pseudo-obstruction (Ogilvie's syndrome), which consists of apparent obstruction at the splenic flexure, although no associated cause is found by barium enema or colonoscopy for the failure of gas and feces to pass.

Unfortunately, many lengthy post operative stays in the hospital are associated with Ileus, where the patient simply cannot be discharged until his bowels move. The clinical consequences of postoperative Ileus can be profound. Patients with Ileus are immobilized, have discomfort and pain, and are at increased risk for pulmonary complications. Ileus also enhances catabolism because of poor nutrition. It has been reported in the 1990's that, overall, Ileus prolongs hospital stays, costing $750 million annually in the United States. Thus, it stands to reason that the healthcare costs associated with Ileus over a decade later are much higher. The relatively high medical costs associated with such post operative hospital stays are clearly undesirable, not to mention patient discomfort, and other complications. There are not, however, any commercially available medical equipment that can treat Ileus. It is therefore desirable to avoid the complications associated with the temporary arrest of intestinal peristalsis, particularly that resulting from abdominal surgery, and provide equipment capable of delivering an internal or external treatment to reduce and/or eliminate the pathological responses that are associated with Ileus.

### SUMMARY OF THE INVENTION

In accordance with one or more embodiments of the present invention, methods and apparatus for treating the temporary arrest of intestinal peristalsis provide for: inducing at least one of an electric current, an electric field and an electromagnetic field in a sympathetic nerve chain of a mammal to modulate and/or block inhibitory nerve signals thereof such that intestinal peristalsis function is at least partially improved.

The electric current, electric field and/or electromagnetic field may be applied to at least one of the celiac ganglia, cervical ganglia, and thoracic ganglia of the sympathetic nerve chain. Alternatively or additionally, the electric current, electric field and/or electromagnetic field may be applied to at least a portion of the splanchnic nerves of the sympathetic nerve chain, and/or the spinal levels from T5 to L2.

The methods and apparatus may further provide for inducing the current and/or field(s) by applying at least one electrical impulse to one or more emitters. The one or more emitters may be disposed at least one of subcutaneously and percutaneously to direct the field(s) to the spinal cord and/or sympathetic nerve chain. The one or more emitters may include at least one of contact electrodes, capacitive coupling electrodes, and inductive coils. Drive signals may be applied to the one or more emitters to produce the at least one impulse and induce the current and/or field(s). The drive signals may include at least one of sine waves, square waves, triangle waves, exponential waves, and complex impulses.

The drive signals inducing the current and/or fields preferably have a frequency, an amplitude, a duty cycle, a pulse width, a pulse shape, etc. selected to influence the therapeutic result, namely modulating some or all of the nerve transmissions in the sympathetic nerve chain. By way of example, the parameters of the drive signal may include a square wave profile having a frequency of about 10 Hz or greater, such as between about 15 Hz to 120 Hz, or between about 25 Hz to about 50 Hz. The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts.

The protocol of one or more embodiments of the present invention may include measuring a response of the patient to the applied current and/or field(s). For example, the digestive muscle activity of the patient may be monitored and the parameters of the drive signal (and thus the induced current and/or fields) may be adjusted to improve the treatment.

Other aspects, features, and advantages of the present invention will be apparent to one skilled in the art from the description herein taken in conjunction with the accompanying drawings.

### DESCRIPTION OF THE DRAWINGS

For the purposes of illustration, there are forms shown in the drawings that are presently preferred, it being understood, however, that the invention is not limited to the precise arrangements and instrumentalities shown.

FIGS. 1-2 are schematic diagrams of the human autonomic nervous system, illustrating sympathetic fibers, spinal nerve root fibers, and cranial nerves;

FIG. 3 is another schematic diagram of the human autonomic nervous system including an apparatus for electrically stimulating, blocking and/or modulating the sympathetic fibers and/or spinal nerve fibers; and

FIG. 5 is a graphical illustration of an electrical signal profile that may be used to treat disorders through neuromuscular modulation in accordance with one or more embodiments of the present invention.

### DETAILED DESCRIPTION OF THE PREFERRED EMBODIMENTS

Ileus occurs from hypomotility of the gastrointestinal tract in the absence of a mechanical bowel obstruction. This suggests that the muscle of the bowel wall is transiently impaired and fails to transport intestinal contents. This lack of coordinated propulsive action leads to the accumulation of both gas and fluids within the bowel. Although Ileus has numerous causes, the postoperative state is the most common scenario for Ileus development. Frequently, Ileus occurs after intraperitoneal operations, but it may also occur after retroperitoneal and extra-abdominal surgery. The longest duration of Ileus has been reported to occur after colonic surgery.

According to some hypotheses, postoperative Ileus is mediated via activation of inhibitory spinal reflex arcs. Anatomically, three distinct reflexes are involved: ultrashort reflexes confined to the bowel wall, short reflexes involving prevertebral ganglia, and long reflexes involving the spinal cord. Spinal anesthesia, abdominal sympathectomy, and nerve-cutting techniques have been demonstrated to either prevent or attenuate the development of Ileus. The surgical stress response leads to systemic generation of endocrine and inflammatory mediators that also promote the development of Ileus. Rat models have shown that laparotomy, eventration, and bowel compression lead to increased numbers of macrophages, monocytes, dendritic cells, T cells, natural killer cells, and mast cells, as demonstrated by immunohistochemistry. Calcitonin gene-related peptide, nitric oxide, vasoactive intestinal peptide, and substance P function as inhibitory neurotransmitters in the bowel nervous system. Nitric oxide and vasoactive intestinal peptide inhibitors and substance P receptor antagonists have been demonstrated to improve gastrointestinal function.

In accordance with one or more embodiments of the present invention, a method of treating a temporary arrest of intestinal peristalsis (such as Ileus) includes inducing an electric current, an electric field and/or an electromagnetic field in a sympathetic nerve chain of a mammal to block inhibitory nerve signals thereof such that intestinal peristalsis function is at least partially improved. The electric current, electric field and/or electromagnetic field may be induced by way of externally disposed apparatus, such as a control unit (including a drive signal generator) and percutaneous and/or subcutaneous emitters, such as contact electrodes, capacitive coupling electrodes and/or inductive coils. (Alternative embodiments of the present invention may provide for entirely subcutaneous components, including the control unit, signal generator, and/or the electrodes/coils).

The emitters (whether disposed percutaneously or subcutaneously) are preferably located to direct the current, electric and/or electromagnetic fields to or toward one or more portions of the spinal cord and/or sympathetic nerve chain. Particular locations for the emitters include one or more areas such that the electric current, electric field and/or electromagnetic field is applied to at least one of the celiac ganglia, cervical ganglia, and thoracic ganglia of the sympathetic nerve chain. Alternative and/or additional locations for the emitters include one or more areas such that the electric current, electric field and/or electromagnetic field is applied to at least a portion of the splanchnic nerves of the sympathetic nerve chain, and/or one or more of the spinal levels from T5 to L2.

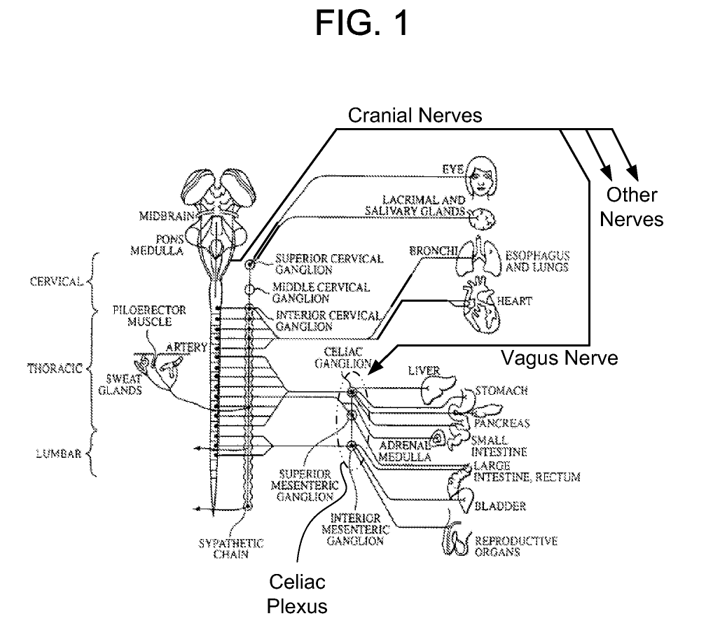

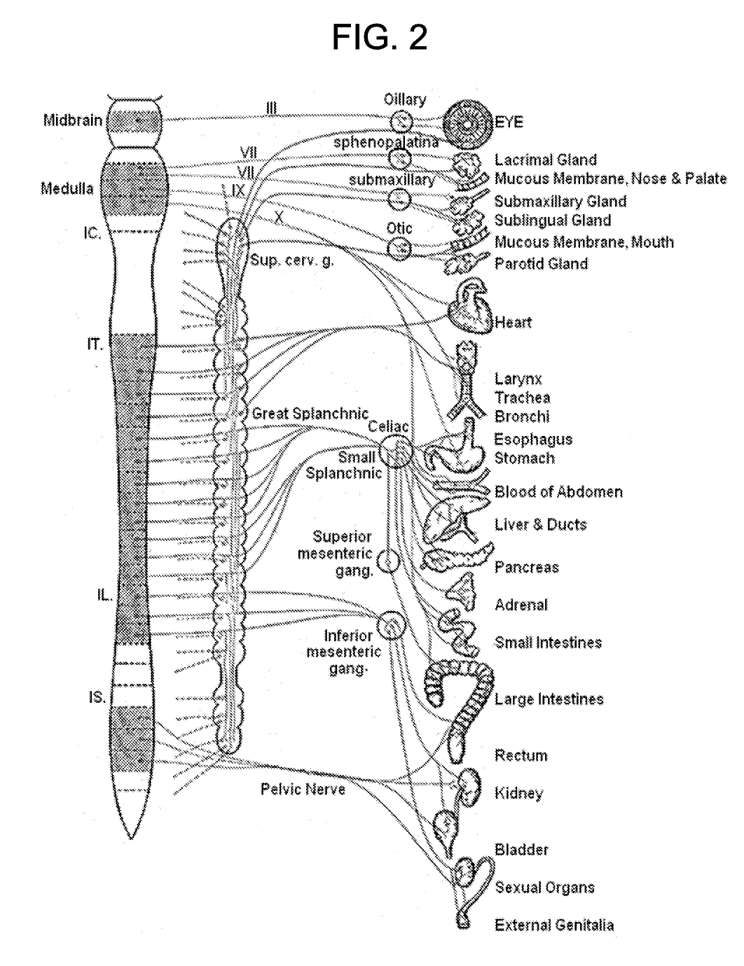

In connection with the location(s) of the emitter(s), a discussion of the human autonomic nervous system, including sympathetic fibers and parasympathetic fibers will now be provided with reference to FIGS. 1-3. The sympathetic nerve fibers, along with many of the spinal cord's nerve root fibers, and the cranial nerves that innervate tissue in the thoracic and abdominal cavities are sometimes referred to as the autonomic, or vegetative, nervous system. The sympathetic, spinal, and cranial nerves all have couplings to the central nervous system, generally in the primitive regions of the brain, however, these components have direct effects over many regions of the brain, including the frontal cortex, thalamus, hypothalamus, hippocampus, and cerebellum. The central components of the spinal cord and the sympathetic nerve chain extend into the periphery of the autonomic nervous system from their cranial base to the coccyx, essentially passing down the entire spinal column, including the cervical, thoracic and lumbar regions. The sympathetic chain extends on the anterior of the column, while the spinal cord components pass through the spinal canal. The cranial nerves, the one most innervating of the rest of the body being the vagus nerve, passes through the dura mater into the neck, and then along the carotid and into the thoracic and abdominal cavities, generally following structures like the esophagus, the aorta, and the stomach wall.

Because the autonomic nervous system has both afferent and efferent components, modulation of its fibers can affect both the end organs (efferent) as well as the brain structure to which the afferents fibers are ultimately coupled within the brain.

Although sympathetic and cranial fibers (axons) transmit impulses producing a wide variety of differing effects, their component neurons are morphologically similar. They are smallish, ovoid, multipolar cells with myelinated axons and a variable number of dendrites. All the fibers form synapses in peripheral ganglia, and the unmyelinated axons of the ganglionic neurons convey impulses to the viscera, vessels and other structures innervated. Because of this arrangement, the axons of the autonomic nerve cells in the nuclei of the cranial nerves, in the thoracolumbar lateral comual cells, and in the gray matter of the sacral spinal segments are termed preganglionic sympathetic nerve fibers, while those of the ganglion cells are termed postganglionic sympathetic nerve fibers. These postganglionic sympathetic nerve fibers converge, in small nodes of nerve cells, called ganglia that lie alongside the vertebral bodies in the neck, chest, and abdomen. The effects of the ganglia as part of the autonomic system are extensive. Their effects range from the control of insulin production, cholesterol production, bile production, satiety, other digestive functions, blood pressure, vascular tone, heart rate, sweat, body heat, blood glucose levels, and sexual arousal.

The parasympathetic group lies predominately in the cranial and cervical region, while the sympathetic group lies predominantly in the lower cervical, and thoracolumbar and sacral regions. The sympathetic peripheral nervous system is comprised of the sympathetic ganglia that are ovoid/bulb like structures (bulbs) and the paravertebral sympathetic chain (cord that connects the bulbs). The sympathetic ganglia include the central ganglia and the collateral ganglia.

The central ganglia are located in the cervical portion, the thoracic portion, the lumbar portion, and the sacral portion. The cervical portion of the sympathetic system includes the superior cervical ganglion, the middle cervical ganglion, and the interior cervical ganglion.

The thoracic portion of the sympathetic system includes twelve ganglia, five upper ganglia and seven lower ganglia. The seven lower ganglia distribute filaments to the aorta, and unite to form the greater, the lesser, and the lowest splanchnic nerves. The greater splanchnic nerve (splanchnicus major) is formed by branches from the fifth to the ninth or tenth thoracic ganglia, but the fibers in the higher roots may be traced upward in the sympathetic trunk as far as the first or second thoracic ganglion. The greater splanchnic nerve descends on the bodies of the vertebrae, perforates the crus of the diaphragm, and ends in the celiac ganglion of the celiac plexus. The lesser splanchnic nerve (splanchnicus minor) is formed by filaments from the ninth and tenth, and sometimes the eleventh thoracic ganglia, and from the cord between them. The lesser splanchnic nerve pierces the diaphragm with the preceding nerve, and joins the aorticorenal ganglion. The lowest splanchnic nerve (splanchnicus imus) arises from the last thoracic ganglion, and, piercing the diaphragm, ends in the renal plexus.

The lumbar portion of the sympathetic system usually includes four lumbar ganglia, connected together by interganglionic cords. The lumbar portion is continuous above, with the thoracic portion beneath the medial lumbocostal arch, and below with the pelvic portion behind the common iliac artery. Gray rami communicantes pass from all the ganglia to the lumbar spinal nerves. The first and second, and sometimes the third, lumbar nerves send white rami communicantes to the corresponding ganglia.

The sacral portion of the sympathetic system is situated in front of the sacrum, medial to the anterior sacral foramina. The sacral portion includes four or five small sacral ganglia, connected together by interganglionic cords, and continuous above with the abdominal portion. Below, the two pelvic sympathetic trunks converge, and end on the front of the coccyx in a small ganglion.

The collateral ganglia include the three great gangliated plexuses, called, the cardiac, the celiac (solar or epigastric), and the hypogastric plexuses. The great plexuses are respectively situated in front of the vertebral column in the thoracic, abdominal, and pelvic regions. They consist of collections of nerves and ganglia; the nerves being derived from the sympathetic trunks and from the cerebrospinal nerves. They distribute branches to the viscera.

The celiac plexus is the largest of the three great sympathetic plexuses and is located at the upper part of the first lumbar vertebra. The celiac plexus is composed of the celiac ganglia and a network of nerve fibers uniting them together. The celiac plexus and the ganglia receive the greater and lesser splanchnic nerves of both sides and some filaments from the right vagus nerve. The celiac plexus gives off numerous secondary plexuses along the neighboring arteries. The upper part of each celiac ganglion is joined by the greater splanchnic nerve, while the lower part, which is segmented off and named the aorticorenal ganglion, receives the lesser splanchnic nerve and gives off the greater part of the renal plexus.

The secondary plexuses associated with the celiac plexus consist of the phrenic, hepatic, lineal, superior gastric, suprarenal, renal, spermatic, superior mesenteric, abdominal aortic, and inferior mesenteric. The phrenic plexus emanates from the upper part of the celiac ganglion and accompanies the inferior phrenic artery to the diaphragm, with some filaments passing to the suprarenal gland and branches going to the inferior vena cava, and the suprarenal and hepatic plexuses. The hepatic plexus emanates from the celiac plexus and receives filaments from the left vagus and right phrenic nerves. The hepatic plexus accompanies the hepatic artery and ramifies upon its branches those of the portal vein in the substance of the liver. Branches from hepatic plexus accompany the hepatic artery, the gastroduodenal artery, and the right gastroepiploic artery along the greater curvature of the stomach.

The lienal plexus is formed from the celiac plexus, the left celiac ganglion, and from the right vagus nerve. The lienal plexus accompanies the lienal artery to the spleen, giving off subsidiary plexuses along the various branches of the artery. The superior gastric plexus accompanies the left gastric artery along the lesser curvature of the stomach, and joins with branches from the left vagus nerve. The suprarenal plexus is formed from the celiac plexus, from the celiac ganglion, and from the phrenic and greater splanchnic nerves. The suprarenal plexus supplies the suprarenal gland. The renal plexus is formed from the celiac plexus, the aorticorenal ganglion, and the aortic plexus, and is joined by the smallest splanchnic nerve. The nerves from the suprarenal plexus accompany the branches of the renal artery into the kidney, the spermatic plexus, and the inferior vena cava.

The spermatic plexus is formed from the renal plexus and aortic plexus. The spermatic plexus accompanies the internal spermatic artery to the testis (in the male) and the ovarian plexus, the ovary, and the uterus (in the female). The superior mesenteric plexus is formed from the lower part of the celiac plexus and receives branches from the right vagus nerve.

The superior mesenteric plexus surrounds the superior mesenteric artery and accompanies it into the mesentery, the pancreas, the small intestine, and the great intestine. The abdominal aortic plexus is formed from the celiac plexus and ganglia, and the lumbar ganglia. The abdominal aortic plexus is situated upon the sides and front of the aorta, between the origins of the superior and inferior mesenteric arteries, and distributes filaments to the inferior vena cava. The inferior mesenteric plexus is formed from the aortic plexus. The inferior mesenteric plexus surrounds the inferior mesenteric artery, the descending and sigmoid parts of the colon and the rectum.

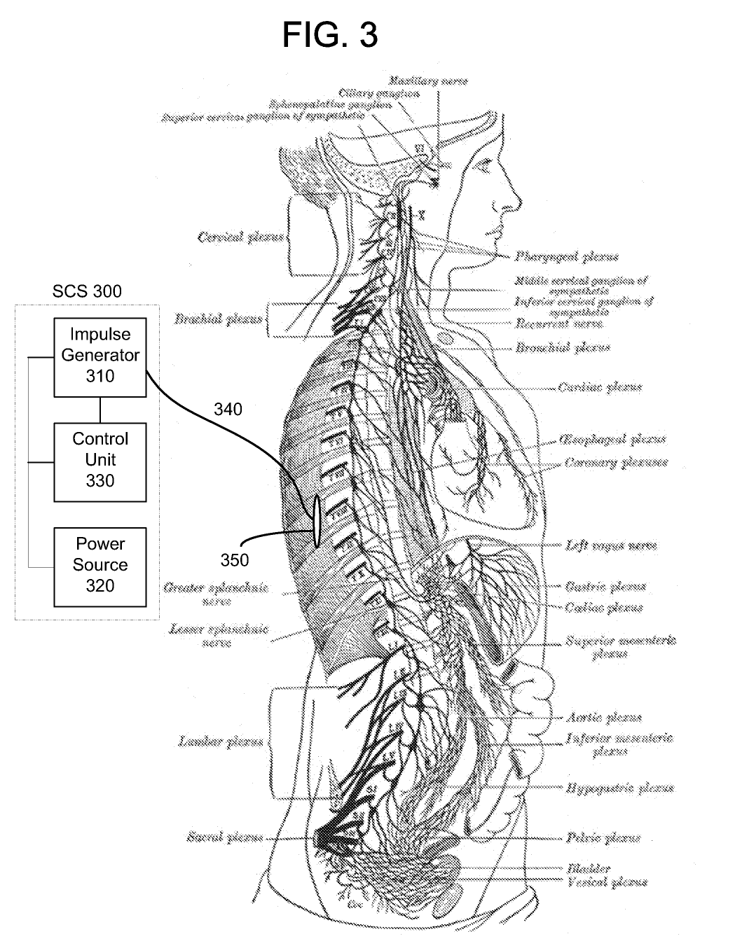

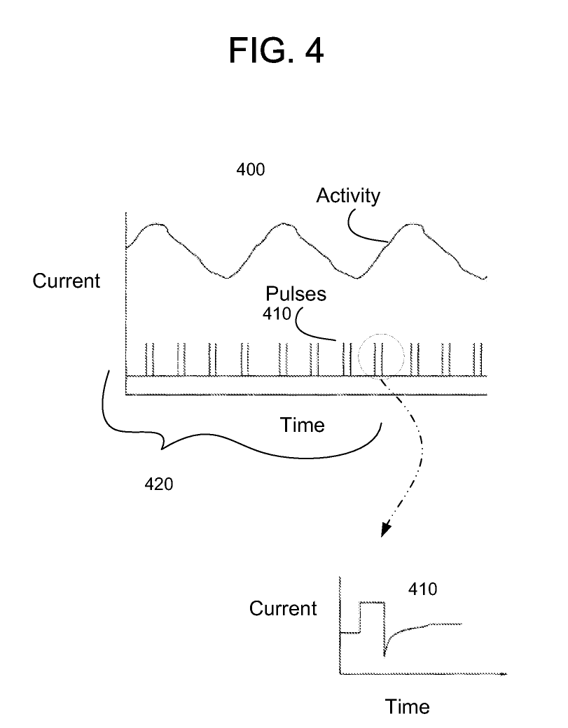

The current and/or fields may be induced by applying at least one electrical impulse to the emitters, such as by using the signal generator to apply the drive signals to the emitters. Particular reference is now made to FIG. 3, which illustrates a view of the anatomy and a spinal cord nerve stimulation device (SCS) 300 for blocking and/or modulating inhibitory nerve signals such that intestinal peristalsis function is at least partially improved. The SCS device 300 may include an electrical impulse generator 310; a power source 320 coupled to the electrical impulse generator 310; a control unit 330 in communication with the electrical impulse generator 310 and coupled to the power source 320; and electrodes 350 coupled to the electrical impulse generator 310 for attachment via leads 340 to one or more selected regions of a mammal. The device 300 may be self-contained or comprised of various separate, interconnected units. The control unit 330 may control the electrical impulse generator 310 for generation of a signal suitable for blocking and/or modulating inhibitory nerve signals when the signal is applied via the electrodes 350 to the nerves. It is noted that SCS device 300 may be referred to by its function as a pulse generator.

By way of example, the drive signals may include at least one of sine waves, square waves, triangle waves, exponential waves, and complex impulses. In one or more embodiments, the signal generator may be implemented using a power source, a processor, a clock, a memory, etc. to produce the aforementioned waveforms, such as a pulse train. The parameters of the drive signal are preferably programmable, such as the frequency, amplitude, duty cycle, pulse width, pulse shape, etc. In the case of an implanted signal generator, programming may take place before or after implantation. For example, an implanted signal generator may have an external device for communication of settings to the generator. An external communication device may modify the signal generator programming to improve treatment.

In the case of contact electrodes, such preferably exhibit impedances permitting a peak pulse voltage in the range from about 0.2 volts to about 20 volts. The blocking and/or modulating signal (current and/or fields) preferably have a frequency, an amplitude, a duty cycle, a pulse width, a pulse shape, etc. selected to influence the therapeutic result, namely blocking some or all of the nerve transmissions in the spinal cord/sympathetic nerve chain.

FIG. 4 illustrates an exemplary electrical voltage/current profile for a blocking and/or modulating inhibitory nerve signals in the sympathetic nerve chain. Application of a suitable electrical voltage/current profile 400 may be achieved using the pulse generator 310. In a preferred embodiment, the pulse generator 310 may be implemented using the power source 320 and the control unit 330 having, for instance, a processor, a clock, a memory, etc., to produce a pulse train 420 to the electrode(s) 350 that deliver the blocking impulses 410 to the sympathetic nerve chain via leads 340.

By way of example, the parameters of the drive signal may include a sine wave profile having a frequency of about 10 Hz or greater, such as between about 15 Hz to 120 Hz, or between about 25 Hz to about 50 Hz. (These are notably higher frequencies than typical nerve stimulation or modulation frequencies.) The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts.

The protocol of one or more embodiments of the present invention may include measuring a response of the patient to the applied current and/or field(s). For example, the digestive muscle activity of the patient may be monitored and the parameters of the drive signal (and thus the induced current and/or fields) may be adjusted to improve the treatment.

Among the available devices to implement the control unit and/or signal generator for facilitating the induced current and/or the emission of electric fields and/or electromagnetic fields is a physician programmer, such as a Model 7432 also available from Medtronic, Inc. An alternative control unit, signal generator is disclosed in U.S. patent Publication No.: 2005/0216062, the entire disclosure of which is incorporated herein by reference. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated. It is noted that if the aforementioned hardware requires modification to achieve the parameters of the drive signals, then one skilled in the art would not require undue experimentation to achieve such modifications, or one skilled in the art would readily be able to obtain hardware capable of producing the drive signals based on the description herein.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating a temporary arrest of intestinal peristalsis, comprising inducing at least one of an electric current, an electric field and an electromagnetic field in a sympathetic nerve chain of a mammal to block and/or modulate inhibitory nerve signals thereof such that intestinal peristalsis function is at least partially improved.

2. The method of claim 1, wherein the electric current, electric field and/or electromagnetic field is applied to at least one of the celiac ganglia, cervical ganglia, and thoracic ganglia of the sympathetic nerve chain.

3. The method of claim 1, wherein the electric current, electric field and/or electromagnetic field is applied to at least a portion of the splanchnic nerves of the sympathetic nerve chain.

4. The method of claim 1, wherein the electric current, electric field and/or electromagnetic field is applied to a spine of the mammal at one or more of the levels from T5 to L2.

5. The method of claim 1, further comprising inducing the current and/or field(s) by applying at least one electrical impulse to one or more emitters.

6. The method of claim 5, wherein the one or more emitters are disposed at least one of subcutaneously and percutaneously to direct the field(s) to the sympathetic nerve chain.

7. The method of claim 6, wherein the one or more emitters include at least one of contact electrodes, capacitive coupling electrodes, and inductive coils.

8. The method of claim 5, further comprising applying drive signals to the one or more emitters to produce the at least one impulse and induce the current and/or field(s).

9. The method of claim 8, wherein the drive signals include at least one of sine waves, square waves, triangle waves, exponential waves, and complex impulses.

10. The method of claim 9, wherein the drive signals include a frequency of about 10 Hz or greater.

11. The method of claim 10, wherein the frequency is between about 15 Hz to 120 Hz.

12. The method of claim 11, wherein the frequency is between about 25 Hz to about 50 Hz.

13. The method of claim 9, wherein the drive signals include a duty cycle of between about 1 to 100%.

14. The method of claim 9, wherein the drive signals include a peak amplitude of about 0.2 volts or greater.

15. The method of claim 14, wherein the peak amplitude is between about 0.2 an about 20 volts.

16. The method of claim 9, wherein the drive signals include a pulse width of about 20 us or greater.

17. The method of claim 16, wherein the pulse width is between about 100 to about 1000 us.

18. The method of claim 1, further comprising measuring a response of the mammal to the current and/or field(s).

19. The method of claim 18, wherein the response includes digestive muscle activity.

20. The method of claim 18, further comprising adjusting at least one parameter of the current and/or field(s) as a function of the response.
